(19) United States
(12) Patent Application Publication
(10) Pub. No.: US 2025/0186652 A1
(43) Pub. Date: Jun. 12, 2025

(54) ARTICLES AND METHODS FOR IMPROVED TISSUE HEALING

(71) Applicant: UNIVERSITY OF MISSISSIPPI MEDICAL CENTER, Jackson, MS (US)

(72) Inventors: Michelle A. TUCCI, Jackson, MS (US); Hamed A. BENGHUZZI, Jackson, MS (US); Robert A. MCGUIRE, Jackson, MS (US)

(21) Appl. No.: 18/844,361

(22) PCT Filed: Mar. 8, 2023
(86) PCT No.: PCT/US2023/014829

§ 371 Date: Sep. 5, 2024

Related U.S. Application Data
(60) Provisional application No. 63/317,841, filed on Mar. 8, 2022.

(51) Int. Cl. A61L 17/00; A61L 15/24; A61L 15/44; A61L 15/64; A61L 17/10

(52) U.S. Cl. CPC A61L 17/005; A61L 15/24; A61L 15/44; A61L 15/64; A61L 17/10; A61L 2300/232; A61L 2300/412; A61L 2430/34

(57) ABSTRACT

A tissue repair article that comprises a material such as a suture or bandage combined with one or more with a wound healing agent; wherein the wound healing agent comprises mannose 6 phosphate (M6P), fructose 6 phosphate (F6P), or glucose 6 phosphate (G6P) or a combination thereof.

### TECHNICAL FIELD

The presently-disclosed subject matter generally relates to articles and methods for improved tissue healing. More specifically, the presently-disclosed subject matter relates to coated sutures for improved tissue healing and methods of making and using the same.

### BACKGROUND AND SUMMARY

While the technical aspects of suture repair of tendon, meniscus, and nerves have been thoroughly studied and refined over the past fifty years, no adjunctive treatments have been shown to provide a significant improvement in healing over repair alone. As a result, clinicians and patients continue to encounter complications such as rupture, decreased range of motion secondary to adhesions, and loss of excursion. These problems are all associated with disorganized or excessive collagen production by fibroblasts.

Accordingly, one embodiment of the present invention is a wound repair material that is coated, impregnated, infused, or otherwise combined with a material that provides for improved wound healing.

In another embodiment, the device is a suture. In one aspect, the suture may be a biodegradable and bioresorbable material that is used for joint repair, particularly with respect to tendon, ligaments, or cartilage. In another aspect, the suture is used to repair incisional wounds. In yet another aspect, the suture is used to repair nerve tissue.

In another aspect, the material that provides for improved wound healing is mannose-6-phosphate.

### Tendon Injuries

### Tendon Therapy

Considerable differences exist in therapy for acute versus chronic injuries to tendon, ranging from over-the-counter medication to physical therapy to surgery, and multiple permutations of these (Andres and Murrell 2008). Treatment is guided by the anatomical location of the tendon, the duration and degree of pain, and patient factors such as comorbidities and physical activity level. Some of the commonly prescribed treatments for tendon injury provide no benefit in the healing process. Instead, they provide symptomatic relief from the pain and inflammation associated with these injuries.

Mild acute tendinopathy is commonly treated with NSAIDs. It is important to note that NSAIDs do not assist in healing. In fact, they may inhibit healing through the reduction of prostaglandins involved in the inflammatory response to injury. However, in mild tendinopathy they are helpful in reducing pain and swelling. NSAIDs function through reversible inhibition of the cyclo-oxygenase enzymes (COX-1 and COX-2), which catalyze the formation of prostaglandins, prostacyclins, and thromboxanes from arachidonic acid (AA). While COX-1 is constitutively produced by many cell types, COX-2 is an inducible isoform of cyclo-oxygenase which is associated with the inflammatory response to injury and sensitization of pain receptors (Mehallo, Drezner et al. 2006). Therefore, this isoform is the primary target for the treatment of mild acute tendinopathy. While selective inhibitors of the COX-2 isoenzyme exist, several have been pulled or banned by the FDA. Most acute tendinopathy is treated with short-acting non-selective COX inhibitors such as ibuprofen or naproxen.

Chronic tendinopathy is frequently treated with steroid injections within or around the tendon parenchyma. Steroids inhibit the production of phospholipase A2, which catalyzes the formation of AA. In macrophages, steroids inhibit transcription and secretion of interleukin-1 (IL-1), IL-6, and tumor necrosis factor (TNF). These effects on the production of prostaglandins and inflammatory cytokines explain how corticosteroids reduce pain, inflammation, and swelling in zones of injury (Townsend and Sabiston 2004). However, injection of corticosteroid in tendons can have detrimental effects in the long-term, especially in cases of multiple injections. In vitro research on human tenocytes treated with dexamethasone revealed negative effects on tenocyte viability, mitotic activity and collagen production (Wong, Tang et al. 2003). Dexamethasone has also been seen to decrease proteoglycan production by human tenocytes (Wong, Tang et al. 2005). A meta-analysis of clinical studies of steroid injections for treatment of tendonitis determined that no long-benefit was found in symptom relief of patients with tendinopathy (Gaujoux-Viala, Dougados et al. 2009, Reddy, Pedowitz et al. 2009).

Debridement of frayed or degenerated tendon is the most commonly employed surgical option for patients suffering with chronic tendinopathy unresponsive to medical management (Reddy, Pedowitz et al. 2009). In some cases when extensive debridement is necessary, augmentation of the tendon is performed with a turn-down flap, transfer of autologous tendon, or synthetic graft incorporation (Grundy, O'Sullivan et al. 2010). Other patients may require release of the fascia cruris and the peritenon (Nelen, Martens et al. 1989). Combined with appropriate physical therapy, surgical treatment of chronic tendinopathy is successful at relieving patients' symptoms and allowing them to return to full activity in approximately six to eight months (Johnston, Scranton et al. 1997).

Nearly all tendinopathy patients undergo a course of physical therapy, usually emphasizing stretching and eccentric strengthening exercises. Both clinical and laboratory studies have reported on the benefits of stretching in tendon healing (Jensen and Di Fabio 1989; Skutek, van Griensven et al. 2001; Stasinopoulos, Stasinopoulos et al. 2010). It has been seen however, that over-stretching causes release of cytokines which increase local inflammation (Skutek, van Griensven et al. 2001). This potential problem is avoided by close therapist oversight of the rehabilitation program.

Due to the frequent inadequacy of healing after tendon injury, it should not be surprising that scientists have sought new methods to improve or hasten the process. The most frequently touted alternative therapies for tendon healing incorporate ultrasound, either as a primary therapy or as a tool for guided injections into the tendon parenchyma. Several studies on rat Achilles tendon healing aided by ultrasound therapy found increased angiogenesis, better collagen bundle organization and alignment, and increased strength compared to controls (Young and Dyson 1990; da Cunha, Parizotto et al. 2001; Yeung, Guo et al. 2006). Ultrasound-guided therapies include: corticosteroid injection, high-volume saline injection, hyperosmolar dextrose injection (prolotherapy), dry needling, autologous blood injection, percutaneous tenotomy, adhesiotomy (brisement), and injection of sclerosant (Mitchell, Lee et al. 2009; Wijesekera, Chew et al. 2010).

### Use of Controlled Release in Tendon Healing

The use of controlled release of growth factors in tendon healing is a relatively novel concept, with only one group of scientists having published on this topic at the time of this writing. In 2000, this group of researchers devised and characterized a fibrin matrix incorporating a HBDS for the purpose of improving nerve regeneration through controlled release of nerve growth factor (NGF) (Sakiyama-Elbert and Hubbell 2000; Sakiyama-Elbert and Hubbell 2000). Later they adapted this system for the controlled release of platelet-derived growth factor-BB (PDGF-BB) to healing tendon (Sakiyama-Elbert, Das et al. 2008).

Using their fibrin-based delivery system, the researchers performed three in vivo studies on dog flexor tendon repair with controlled release of PDGF-BB (Gelberman, Thomopoulos et al. 2007; Thomopoulos, Zaegel et al. 2007; Thomopoulos, Das et al. 2009). In all three studies, the researchers divided two digital flexor tendons in each dog and placed their delivery system in between the free ends prior to performing a double-loop locking repair. The first study analyzed tendons at days seven and fourteen post-repair by immunohistochemistry for Type I collagen and high performance liquid chromatography (HPLC) for reducible collagen crosslink. At day seven, HPLC revealed significantly increased collagen remodeling in the repaired tendons with the controlled release system compared to those with repair alone. This difference was not seen on day fourteen, suggesting that the controlled release of PDGF-BB allowed tendons to progress to the remodeling phase of healing more quickly.

In the second study, animals were postoperatively treated with continuous passive motion for two five-minute sessions per day for five days starting on post-operative day one. This was continued through the time of sacrifice, three weeks after the operation. The harvested limbs were analyzed for range of motion (ROM) and biomechanical properties. The group treated with controlled release of PDGF-BB had significantly greater ROM than operated controls. There was no significant difference between the controlled release group and the unoperated controls. No significant differences were found in biomechanical parameters between the controlled release group and the operated controls. In the third study, the authors extended their analysis to forty-two days, but obtained similar results to those in the second study. They felt the lack of difference in biomechanical parameters of strength could be attributed to suboptimal release kinetics of their delivery system.

Most recently, this same group has published an in vitro study comparing the release characteristics of basic fibroblast growth factor (bFGF) and PDGF-BB from their delivery system (Thomopoulos, Das et al. 2010). While they found a slower release of bFGF than PDGF-BB from their HBDS, both growth factors were shown to stimulate fibroblast proliferation. Real-time quantitative PCR for both growth factors revealed down-regulation of Type I and Type III collagen and up-regulation of MMP 1 and 13, which are collagen degradation enzymes.

### Meniscal Injuries

Meniscal injuries are the most common reason for arthroscopic knee surgery in the United States. In 2006, approximately 900,000 arthroscopic knee surgeries were performed in the United States. In greater than 50% of the procedures, a meniscal tear was identified (Kim, 2011). Historically, meniscal injuries were treated with meniscectomy, but long term studies have shown that this leads to the development of early osteoarthritis and long term morbidity for the patient (Laible, 2013). Over the past two decades, the focus has turned to the development of meniscal preservation techniques in order to delay or prevent the development of early osteoarthritis.

### Meniscus Anatomy and Function

The medial and lateral meniscus are a vital component of the tibiofemoral joint. Their primary function is to provide a load bearing surface and shock absorption during ambulation. It increases the congruency of the knee joint, which leads to increased contact area and decreased point loading. Studies have shown that point loading pressure is increased in total meniscectomies due to a 50-70% decrease in surface contact (Ahmed, 1983). The meniscus transmits approximately 50% of the weight bearing load in extension, and approximately 85% during flexion. A secondary function of the menisci is joint stability, specifically anterior translation of the tibia. The meniscus is a crescent shaped fibrocartilaginous structure. It is composed of 70% water and 30% organic matter. The organic matter is primarily Type I collagen, but other types do exist. Other organic matter present includes: proteoglycans, DNA, and elastin.

The arrangement of the collagen fibers is important to the force dissipation by the meniscus. The fibers are arranged in a variety of patterns. Circumferentially oriented fibers function to disperse hoop stresses. The compressive forces on the articular surface are translated into concentric forces (hoop forces) on the meniscus. The circumferential fibers aid in distributing these forces to the tibia through the bony anchors of the meniscus. This function protects the articular cartilage from damage. Radially oriented fibers function to resist longitudinal tearing of the meniscus. Finally, randomly oriented fibers on the meniscal surface help to disperse sheer stress during knee flexion.

The meniscus is known to have a very poor blood supply (Arnoczky, 1982). The main blood supply is from the medial and lateral genicular arteries. Only the peripheral 20-30% of the medial meniscus and 10-25% of the lateral meniscus is vascularized. The remaining area is provided nutrition through diffusion. The vascular supply has been divided into three “vascular zones”. These zones include: red-red, red-white, and white-white zones. The red-red zone is contained completely within the vascularized portion of the meniscus, and is considered to have the highest healing potential. The red-white zone is partially contained within the vascularized portion of the meniscus, and has less predictable healing. The red-red and red-white zones are within the outer 4 mm of the meniscus periphery. The white-white zone is the avascular zone. This zone has poor to no healing response, and most of the nutrition is received via diffusion from the synovial fluid. Cooper et al. has expanded upon this zoning scheme, and has described 12 different zones of the meniscus.

### Meniscal Repair Techniques

Meniscal tears are one of the most common injuries to the knee joint, and they often necessitate surgery. Specifically, in the younger, active patient with an acute meniscal injury. Traumatic or acute meniscal injuries occur more frequently in a younger population due to sports-related injuries. Degenerative or chronic tears typically occur in older patients and have an insidious onset. In younger individuals, a healthy meniscus is vital to maintaining the articular cartilage of the tibia and femur. The medial meniscus is injured at a rate three times more frequent than the lateral meniscus. The medial meniscus has more soft tissue attachment than the lateral meniscus, which make it less mobile. The decreased mobility of the medial meniscus likely accounts for the increased rate of injury. Despite this theory, the lateral meniscus is torn more frequently with anterior cruciate ligament injuries.

Meniscal tears are classified based on three characteristics: chronicity of the tear, location in relation to the vascular zone, the anatomic zone of the meniscus, and the appearance and orientation. The types of tear include: horizontal tears, longitudinal tears, radial tears, parrot beak tears, root tears, and bucket handle tears. The location within the corresponding vascular zone of the meniscus is the most important determinant of healing potential. Meniscal tears within 2 mm of the periphery have the highest potential for healing. These tears are within the red-red and red-white zones of the meniscus. On the other hand, meniscal tears which occur greater than 4 mm from the periphery have the lowest potential of healing. These tears are within the white-white zone of the meniscus (Scott, 1986; Cannon 1992).

The treatment of meniscal tears includes conservative and/or surgical treatment. Meniscal tears which do not result in intermittent swelling, catching, locking, or giving way can typically be treated without surgical intervention. Treatment includes activity modification, anti-inflammatory medication, physical therapy, and knee injections. Once a patient has failed conservative measures or meets indications surgical intervention can be chosen. Available options for surgical intervention include: total meniscectomies, partial meniscectomies, meniscal repair, and meniscal transplantation.

Historically, symptomatic meniscal tears were treated with total meniscectomy, but this was shown to have long-term morbidity with the early onset of osteoarthritis. Recently, the focus has turned to meniscal preservation techniques in the appropriate patient population. Meniscal tears which aren't amendable to repair are typically treated with partial meniscectomy. These tears include partial thickness tears, tears which are less than 5-10 mm in length, and those that cannot be displaced greater than 1-2 mm (Tengrootenhuysen, 2011; Noyes, 2002). Complex, degenerative, central and radial tears are typically treated with partial meniscectomy. A recent study evaluated the mechanics as a result of a partial meniscectomy, and found that there was a significant increase in peak pressures and mean contact pressure (Bedi, 2010).

Expert panels agree that meniscal repair should be attempted whenever possible, except in the presence of high grade chondral injury. The indications for meniscal repair include: tears within 1-4 cm in length, vertical tears, red-red tears, meniscal root tears, patient age less than 40, no mechanical axis malalignment, acute tears (<6 weeks), and concomitant ACL reconstruction (Scott, 1986; Cannon, 1992; Allaire, 2008). There are four commonly used meniscal repair techniques: “open”, “outside-in”, “inside-out”, and “all-inside”. The gold standard repair technique remains the inside-out technique with vertical mattress sutures. The success rate of meniscal repair has ranged from an 80-90% success rate, but these studies typically have selected patients with the highest healing potential (i.e. red-red vascular zone).

### Meniscal Repair Augmentation

Efforts to improve meniscal repair outcomes have begun to focus on augmentation of the repair. Due to the high success rate of healing with more peripheral tears, repair augmentation has focused on more central meniscal repairs with poor healing potential and less successful outcomes. Methods of repair enhancement have been performed through direct mechanical stimulation and direct placement of a substance or material. The theory of meniscal repair augmentation is to provide a biologically active substance at the repair site in hopes of increasing the healing potential of the avascular meniscal tear.

Biologic Augmentation-Mesenchymal Stem Cells (MSCs) have been explored for meniscal repair augmentation. MSCs have shown some success with meniscal repair involving the avascular zone of the meniscus in animal models (Angele, 2008; Zellner, 2013; Zellner, 2010). The exact mechanisms of MSCs repair augmentation is still unclear. It has been hypothesized that the MSCs serve as the repairing cells themselves, but it has been shown that the growth factors that are provided by the MSCs promote regeneration (Caplan, 2006). Zellner et al study how individual growth factors were able to mimic the effects of MSCs on avascular meniscal tears. The study analyzed the effects of platelet rich plasma, which supplies a number of growth factors, and a single growth factor BMP-7 on avascular meniscal tears. The study showed that PRP and BMP-7 showed positive aspects of meniscal regeneration, but failed to significantly improve healing in the avascular zone of the meniscus. The possible reason for failure was due to uncontrolled release of growth factors in vivo. It was concluded that biological augmentation for meniscal regeneration did seem possible (Zellner, 2014).

Fibrin clots have been used to augment meniscal repairs. The clot is formed by spinning a patient's blood until a clot is formed. The clot is then collected and placed directly onto the repair site to augment the meniscal repair. The placement of a fibrin clot into an isolated meniscal tear resulted in a failure rate of 8%, which was found to be significantly differently than 41% failure rate without the presence of the fibrin clot (Henning, 1990).

Recently, tissue engineering strategies have focused on the manufacturing of biological scaffolds enhanced with cells and growth factors in hopes of increasing the healing response (Moran, 2015; Yu, 2015). Scaffolds consisting of biodegradable polymers have been shown to mimic meniscal tissues resulting in differentiation of mesenchymal stem cells into fibrochondrogenic pathways (Baker, 2010). Multilayered scaffold sheets incorporated into meniscal tears have shown to mimic the biomechanical properties of the native meniscus (Rothrauff, 2016). Monibi et al studied the development of decellularized canine menisci to fabricate a micronized, ECM-derived scaffold and to determine the scaffolds meniscal repair potential ex vivo. They compared radial meniscus tears augmented with the scaffold delivered with platelet-rich-plasma to non-augmented suture repair. Histologically, there was no cellular migration or proliferation noted in the suture repair group. The scaffold group showed cellular infiltration and proliferation (Monibi, 2016). An injectable extracellular matrix hydrogel has also been developed for meniscal repair augmentation. An injectable ECM was development to overcome the limitations of and ECM scaffold, which were poor cell infiltration and implantation via a surgical procedure. The hydrogel showed good cellular compatibility by promoting growth of bovine chondrocytes and mouse 3T3 fibroblasts (Wu, 2014).

Marrow Stimulating Augmentation-Meniscal repair in the setting of concomitant ACL reconstruction has shown superior outcomes compared to isolated meniscal repairs. In theory, the reasoning behind these improved outcomes is due to the drilling of the ACL graft tunnel. These releases a number of intramedullary release of peptides, growth factors, and pluripotent cells from the bone marrow. This creates a favorable environment for meniscal healing. In order to recreate this environment marrow stimulating procedures have been explored to augment the meniscal repair (Dean, 2017). Mechanical stimulation has been shown to increase cytokines at the meniscal repair site, which promote the healing response (Ochi, 2001). Uchio et al showed complete healing in 71% of meniscal tears treated with mechanical stimulation, but the repairs were performed with an ACL reconstruction. Trephination is another mechanical stimulation type technique. It involves creating multiple poke holes within the meniscus using a spinal needle. In theory, this should enhance the flow blood to the avascular zones of the meniscus. An animal model study showed that there was an increase in activity of fibrochondrocytes resulting in fibrovascular tissue (Zhang, 1995). Ahn et al retrospectively evaluated the use of a marrow stimulating technique. A cannulated reamer was used to make a 5-mm diameter hole with a depth of 20-mm into the medullary bone of the intercondylar notch to stimulate bleeding. The study showed increased activity scores and evidence of meniscal healing in the avascular zone (Ahn, 2015). Dean et al studied the effects of a marrow venting procedure on meniscal repair using two cohorts: meniscal repair with marrow venting procedure and meniscal repair with concomitant ACL reconstruction. The study found that there was no difference in outcomes of meniscal healing between the two groups (Dean, 2017).

### Nerve Injury and Repair and Neuroma Formation

Several studies have shown that growth factors and cellular signaling play an important role during nerve repair. Following traumatic peripheral nerve injury, distal axon experience Wallerian degeneration, then cellular signaling is induced to allow axons to begin to reconstruct the nerve. The generation capacity of the axons and growth support of Schwann cells begins to decline with time and an unorganized axon regrowth patterns begins to develop. Simultaneous proliferation along with signaling molecule upregulation leads to collagen remodeling and scar formation that develops into a poorly vascularized dense fibrous structure known as the neuroma. Inflammatory signaling along with infiltration of myofibroblast are the key components found in painful neuromas.

Treatments for neuromas include surgical and physical modalities, medicine, and neurolytic approaches. Nonsurgical techniques including neuropathic medications, topical or injectable anesthetics (Chabal et al., 1992), radiofrequency ablation (Restrepo-Garces et al., 2011), and chemical axonotmesis (Gruber et al., 2008) have been inconsistent in relieving neuroma pain. Medical interventions for limb pain resulting from neuromas have utilized nonsteroidal anti-inflammatory drugs, tricyclic anti-depressants, and anti-convulsants with limited success (World Health Organization, 2006). Most other medicine regimens have been the subject of sporadic case reports or case series and have not been shown to be effective. Minimally invasive neurolysis with the use of phenol and cryo nerve blocks have been shown to be partially effective in the pain control for neuroma. Radio frequency neurolysis is usually effective for three to five months with longer results reported on repeated procedures. Physical modalities such as massage, ultrasound, vibration, percussion, acupuncture, and modification of the socket for pressure relief have had very limited success in reducing pain due to neuroma. Transcutaneous electrical stimulation (TENS) is widely used and reports suggest temporary pain relief in 50% of patients (Wiffin et al., 2006). Surgical treatments including nerve transposition to healthy bone, vein, or muscle, nerve capping, and traction neurectomy (Robbins, 1986; Gonzales-Darder et al., 1985; Swanson et al., 1977; Dorsi et al., 2008; Chiu et al., 2013) have been shown to reduce pain; however, symptoms can reoccur (Balcin, 2009; Chiu 2013). A common approach to treating painful neuromas is to bury the nerve stump in a healthy muscle. While this technique repositions the stump, neuroma can redevelop in a protected location, theoretically reducing mechanical pain. However, pain symptoms often return. A recent outcome study of upper extremity amputations found DASH scores following nerve transposition or the use of simple resection were not significantly different from pre-surgery scores (Guse and Moran, 2013). Resection alone was associated with an unacceptable recurrence rate, and the authors concluded that resection should be discouraged as treatment for upper extremity neuromas. Guse and Moran's data shows surgical procedures, neuroma size, and the severity of preoperative pain all adversely impact the success of surgical intervention. Currently, there is no consensus for treating neuroma pain.

In view of the discussion above, there remains a need in the art for improved healing in such tissue. With that in mind, the present inventors recognized that competitive interference with the action of TGF-β on fibroblast may provide benefits for treatment of injured tendons. In addition, the present inventors recognized that M6P may provide some benefits for treatment of injured tendons through competitive interference with the actions of TGF-β on fibroblasts. Tissue response to M6P may not be dose-dependent, but might require a threshold concentration for benefits to be seen. Evidence in the literature shows that tissue response to TGF-β takes days, and therefore simple coating of the tendon wound with M6P may be of little benefit as most of the treatment will be cleared by the body in a few hours. If the TGF-β-induced production of unorganized Type I collagen (scar tissue) by fibroblasts can be suppressed for the first few days after injury, then IGF-1-induced production of organized Type I collagen by tenocytes will take control. A higher proportion of organized Type I collagen would more closely resemble uninjured tendon and increase the tendon's overall strength and resistance to rupture.

In view thereof, disclosed herein is sustained delivery of M6P to a tendon wound, which is a novel concept that may provide long-term healing benefits to the patient through reduction of poorly organized collagen deposition around the healing tendon. By this mechanism M6P may reduce the formation of adhesions, which can impair range of motion.

One embodiment of the present invention is a tissue repair article that comprises a material that is combined with one or more with a wound healing agents. In aspects of the invention, the wound healing agent comprises mannose 6 phosphate (M6P), fructose 6 phosphate (F6P), or glucose 6 phosphate (G6P) or a combination thereof; or a sugar precursor of mannose 6 phosphate (M6P), fructose 6 phosphate (F6P), or glucose 6 phosphate (G6P) or a combination thereof.

In one embodiment, the material is a surgical suture. In aspects of this embodiment, the suture is coated with the wound healing agent. Also, the suture may be impregnated with the wound healing agent.

In another embodiment of the present invention, the suture is is absorbable when surgically implanted in a subject.

The specific nature of the suture is not known to be critical. Thus, in embodiments of the invention, the suture may be silk, gut, polypropylene, PDS, poliglecaprone, polyglactin, polyester, steel, monofilament, multifilament, polytetrafluoroethylene, poly (DL-lactide-ε-caprolactone, polyester/polyethylene (PE/PEE).

In other embodiments of the invention, the wound healing agent comprises mannose 6 phosphate (M6P), fructose 6 phosphate (F6P).

In other embodiments of the invention, the wound healing agent further comprises at least one liposome, amphiphilic polymer, protein, polycaprolactone (PLA), polyglycolide (PGA), mannuronic acid, hydrogel, a hydrogel derived from alginate (seaweed).

In another embodiment of the invention, the suture is an incisional wound suture.

In another embodiment of the invention, the material is an incisional wound suture and the wound healing agent further comprises mannose.

In another embodiment of the invention, the material is a bandage. The specific nature of the bandage is not known to be critical. For example, in aspects of the invention, the bandage may be a cloth, gauze, or an adhesive bandage. In aspects of the invention, the wound healing agent that is coated on, or impregnated in, the bandage, further comprises at least one liposome, amphiphilic polymer, protein, polycaprolactone (PLA), polyglycolide (PGA), mannuronic acid, hydrogel, a hydrogel derived from alginate (seaweed), or an analgesic.

In aspects of the invention, the material is a natural or synthetic woven fabric, or steel mesh. For example, the material may be a multifilamentous polypropylene mesh.

In another embodiment of the invention, the wound healing agent exhibits sustained release when in contract with a wound site.

In yet another embodiment of the invention, the suture or bandage is coated with a biodegradable layer, and the biodegradable layer is associated with the wound healing agent.

In another embodiment of the invention, the wound healing agent is present in an amount of about 25 to 70 mM, preferably about 50 mM. Also, the wound healing agent may be present in an amount to provide at least three days of sustained release when in contact with an injury site. Also, the wound healing agent is present in amount to effectively inhibit TGF-β at a wound site.

Another aspect of the present invention is a method of promoting tissue healing. Aspects of this embodiment comprise providing a material combined with one or more with a wound healing agents; wherein the wound healing agent comprises mannose 6 phosphate (M6P), fructose 6 phosphate (F6P), or glucose 6 phosphate (G6P) or a combination thereof; or a sugar precursor of mannose 6 phosphate (M6P), fructose 6 phosphate (F6P), or glucose 6 phosphate (G6P) or a combination thereof; and contacting the material to an injury site. The material may be a suture or a bandage. In aspects of this embodiment, the injury is a tendon injury, meniscal injury, nerve injury, or skin injury.

Other aspects of the invention would be obvious to one or ordinary skill in the art when considering the instant disclosure and drawings.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 shows a schematic of the first stage of glycolysis illustrating the isomerization of mannose 6 phosphate (M6P) to fructose 6 phosphate (F6P).

FIG. 2 shows graphs illustrating physical properties of sutures.

FIGS. 3A-D show representative photomicrographs of H&E stained rat Achilles tendons illustrating healing of PBS and M6P groups. (A) Control (400× magnification). (B) Phosphate buffered saline (PBS) group at 2 weeks (left, 100×) and 4 weeks (right, 400×) of repair. (C) M6P direct group at 2 weeks (left, 400×) and 4 weeks (right, 400×) of repair. (D) M6P sustained delivery group at 2 weeks (left, 400×) and 4 weeks (right, 400×) of repair. Brackets represent approximate zones of healing.

FIGS. 4A-D show images illustrating healing of menisci with bucket handle tears treated with various coated sutures. (A) Meniscus with bucket handle tears treated with PBS coated sutures and harvested 14 days after being placed in an organ culture environment. The cut seen is from the cut side just to the left of suture to 3 cm past the suture and sectioning in towards the suture. (B) Menisci with bucket handle tears treated with M6P coated sutures and harvested 14 days after being placed in an organ culture environment. The cuts seen are from just to the left of suture to 3 cm past the suture and sectioning in towards the suture. (C) Only failure from the group of menisci treated with M6P coated sutures. (D) Sections showing comparison in gap width between menisci with bucket handle tears treated with PBS coated sutures and the single failure from the M6P coated sutures group (FIG. 4C). All samples were harvested 14 days after being placed in an organ culture environment.

FIG. 5 shows photographs demonstrating that gut sutures coated with M6P provide a similar tissue response.

FIGS. 6A-B show images illustrating representative sciatic nerve ligation after 4 weeks of constriction using either saline coated or M6P coated sutures and comparing to normal nerve fiber. (A) Gross visualization of tissue response showing limited tissue formation around the suture material as compared to saline coated sutures. (B) Histological comparison of tissue showing there is less sprouting of the nerve and improved mechanical threshold representing pain associated with the nerve in the nerves in contact with the M6P coated sutures.

FIG. 7 shows images illustrating cellular viability and lifting off of the coverslip between M6P (top) and TGF-β1 (bottom) after 48 hours.

FIGS. 8A-B show images illustrating that M6P competes with TGFβ. (A) Use of MRC-5 fibroblast cells in an in vitro assessment of concentration effects of TGFβ and M6P as well as effectiveness of M6P to block the proliferative effects of TGFβ. (B) Schematic showing potential mechanism of M6P and TGFβ competing for the same M6P receptor that leads to increase gene regulation.

FIGS. 9A-B show graphs illustrating release of M6P from tri-calcium phosphate lysine (TCPL) drug delivery device. (A) Gross and net M6P release profile. (B) Cumulative M6P release.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites, and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

When open-ended terms such as “including” or ‘including, but not limited to” are used, there may be other non-enumerated members of a list that would be suitable for the making, using or sale of any embodiment thereof.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

Provided herein are articles that provide improved tissue healing. In some embodiments, the article includes suture materials that are coated, impregnated, or otherwise combined with one or more suitable substances that provide improved tissue healing. Suitable substances include, but are not limited to, mannose 6 phosphate (M6P), fructose 6 phosphate (F6P), glucose 6 phosphate (G6P), sugar precursors thereof (e.g., mannose, D-mannose, fructose, glucose), solutions or hydrogels including the same, or a combination thereof. For example, in some embodiments, the suture material is coated with M6P. In some embodiments, the suture material is coated with F6P. In some embodiments, the suture material is coated with G6P.

Any suitable suture material may be combined with the one or more substances, such as, but not limited to, silk, gut, polypropylene, PDS, poliglecaprone, polyglactin, polyester, steel, monofilament, multifilament, polytetrafluoroethylene, poly (DL-lactide-ε-caprolactone, polyester/polyethylene (PE/PEE).

In another aspect of the invention, the suture material may be sutures for incisional wounds. In this aspect, mannose may be optionally added to the sugar phosphate chosen, such as M6P. Without being bound by theory or mechanism, the use of mannose in the incisional wounds would interfere with the hyaluronic acid and limit the swelling and invasion of epithelial cells to produce a more natural (no scarring) wound, as well as provide a pool for additional Mannose 6 phosphate in the cell microenvironment that would further limit scar and adhesion. Following injury there is a rapid increase of hyaluronic acids in wounds and is associated with tissue swelling, epithelial cell migration, cell proliferation, and inflammatory cytokine. The goal of the hyaluronan is to act as a cable to trap leukocytes and platelets and modulate inflammation. Mannose is an inhibitor of hyaluronic acid and ultimately the fibroblast invasion

Although described herein primarily with respect to suture materials, the article is not so limited and may include any other suitable material used for treatment or healing. Other suitable materials include, but are not limited to, bandages (e.g., cloth, gauze, adhesive, etc.), multifilamentous polypropylene mesh for hernia repair, steel meshes for disc repair, nerve wrap, or on any other natural or synthetic woven fabric material that is placed over a wound to keep it clean.

Also provided herein are methods of making the articles. In some embodiments, the method includes dip coating and vacuum drying the article to reliably place a specific concentration of the sugar phosphates ready for delivery, or synthesizing one or more substance and then extruding the synthesized substance with the article material. For example, in one embodiment, the method includes enzymatically converting mannose to M6P and then extruding the synthesized M6P with the article material. Similarly, fructose or glucose can be enzymatically converted to F6P or G6P, respectively, and then extruded with the article material.

In some embodiments, the sugar/sugar phosphate agent is applied to the suture by impregnating with the sugar/sugar phosphate, and drying the impregnated suture which leaves a sugar/sugar phosphate residue which is distributed throughout the suture structure.

The suture may be impregnated with sugar/sugar phosphate solution by any convenient method such as dipping, spraying, soaking, vacuum impregnation. The material may be dried in a warm oven, under continuous hot air, or any other convenient drying method. The drying temperature may be between 35-100° C. A preferred method is to coat the sutures in an impregnating bath or by vacuum impregnation and followed by drying in warm air.

Additionally or alternatively, the article material may be coated with liposomes, amphiphilic polymers, proteins, polycaprolactone (PLA), polyglycolide (PGA), mannuronic acid, hydrogels, including hydrogels derived from alginate (seaweed). Alginate is a linear polysaccharide that is composed of b-d-mannuronic acid and I-guluronic acid.

Depending upon the manner in which the substance is incorporated into the material, in some embodiments, the article provides sustained release of the substance when in contact with tissue. For example, in one embodiment, the sustained release is provided by enzymatic reaction. In another embodiment, the sustained release is provided by hydrolysis.

Further provided herein are methods of improving tissue healing. In some embodiments, the method includes suturing any suitable tissue using the sutures according to one or more of the embodiments disclosed herein. In some embodiments, the method includes wrapping any suitable tissue using the article according to one or more of the embodiments disclosed herein (e.g., bandage, wrap). In some embodiments, the method includes covering any suitable tissue using the article according to one or more of the embodiments disclosed herein (e.g., bandage). In some embodiments, the method includes supporting any suitable tissue using the article according to one or more of the embodiments disclosed herein (e.g., hernia mesh, bandage, wrap). Suitable tissue includes, but is not limited to, tendon, meniscus, nerve, any tissue that is susceptible to adhesion formation, or any other tissue that experiences difficulty healing following injury.

It is possible since that different wounds heal at different times the bandages the sugar/sugar phosphate can be formulated by direct incorporation into the bandage material or the release can be controlled by using at least one biopolymer, such as polyglycolide (PGA) and polycaprolactone (PLA), to encapsulate the coating after drying. Linear coating of sugars may have varying number of hydroxyl groups which can be used as a conjugation site for polymers such as PLA and PGA.

Additionally, the compounds can be placed at specific ratios into a hydrogel coating that can be applied directly to the bandage to for slow release of the compound over time to cover the wound bed.

Without wishing to be bound by theory, it is believed that the direct and/or sustained administration of the one or more substances in, to, or near the wound provides multiple benefits. In some embodiments, for example, the substance enhances formation of normal tissue. In some embodiments, the substance reduces adhesion formation. More specifically, as shown in the Examples below, the substances disclosed herein compete with TGFβ, which allows for healing with limited fibrosis or scar tissue formation. This reduced or eliminated fibrosis, in turn, reduces the tension from scar tissue formation, which reduces pain and other related complications (e.g., gait, toe spread, etc.). In some embodiments, the substance reduces nerve sprouting. In some embodiments, the substance provides antibiotic activity. Moreover, as opposed to directly coating the tissue, which results in diffusion or clearance of the substance and lack of improved healing, the slower release kinetics and/or sustained release of the substance from the article provides the extended tissue exposure necessary for improved healing. Additionally or alternatively, having the material in close contact with the tissue allows for diffusion into the tissue from the article or direct interaction on the compounds at specific receptors on the surface.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

Many different animal models have been used for studies of orthopedic injuries, including the rat, rabbit, turkey, horse, pig, cat, and dog. The most commonly used animal model for tendon healing in the orthopedic literature is the rat Achilles tendon. While only about one centimeter long by 3 millimeters wide, the rat Achilles tendon is large enough to perform a suture repair after transection. In addition, the large number of animals needed to show a significant difference in the biomechanical parameters favors a small animal model. The pig meniscus is another well studied animal for various repair techniques and healing.

How does M6P reduce fibrosis?

Mannose-6-phosphate, Connective Tissue Growth Factor (CTGF), and Collagen Production

CTGF is a member of the CCN (connective tissue growth factor, cysteine rich protein, and nephroblastoma over-expressed gene) family of regulatory proteins, which along with TGF-β, plays a central role in Type I collagen and ECM production (Quan, Shao et al. 2010). Over-expression of CTGF has been noted in multiple fibrotic tissues (Matsui and Sadoshima 2004; Clavel, Barragan-Montero et al. 2005; Chen, Qi et al. 2009; Dessein, Chevillard et al. 2009). This heightened expression of CTGF may be prompted by placing tissues under hypoxic conditions (Higgins, Biju et al. 2004).

TGF-β plays a role in fibrosis through induction of ECM fabrication by mesenchymal cells, and is additionally recognized to play an important role in signaling for CTGF (Sonnylal, Shi-Wen et al. 2010). CTGF has been shown to further stimulate TGF-β-mediated ECM production (Ihn 2002). This cooperative role of TGF-β and CTGF in fibrotic pathways presents a viable target for therapy in tissue fibrosis.

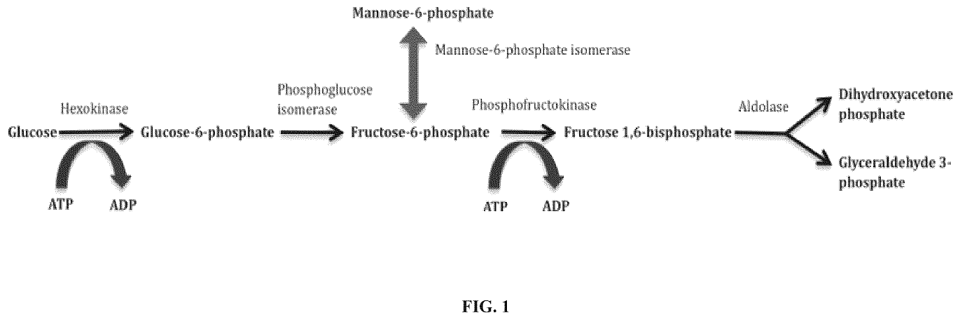

Fructose-1,6-bisphosphate (FBP) is an intermediate in glycolysis which, if provided to cells could allow them to bypass two ATP-requiring steps for energy production. Huang has previously shown that treatment of cells placed under hypoxic conditions with FBP decreases production of CTGF to near control conditions (Haung, Adah et al. 2009). M6P is known to isomerize to fructose-6-phosphate (F6P), an intermediate of glycolysis in the step prior to FBP (FIG. 1). Therefore, one might hypothesize that treatment with M6P may decrease production of CTGF by decreasing the need for substrate-level phosphorylation, which would be vital in hypoxic conditions. In this regard, the present inventors have shown that F6P produces the inhibition of Hif-1 alpha leading to CTGF in vitro. Alternatively, latent TGF-β is activated through its binding with the M6P/IGFII receptor (Ghahary, Tredget et al. 1999). Competitive inhibition of the activation of TGF-β through treatment with M6P is considered another method by which M6P may inhibit fibrosis. It is thought that M6P may have more potential for reducing fibrosis than FBP because its mechanism of action is not dependent upon a hypoxic environment.

Several authors have analyzed the mechanism of action of M6P and its effects on fibroblasts. It has been seen to be a potent inhibitor of human dermal fibroblast and keratinocyte proliferation (Clavel, Barragan-Montero et al. 2005). Greupink et al. used M6P-modified human serum albumin as a hepatic stellate cell-selective drug carrier for mycophenolic acid. They found their delivery system to be effective at decreasing fibrosis of the liver (Greupink, Bakker et al. 2005). The same group later studied how their delivery system specifically targeted the M6P/IGFII receptor in rats subjected to bile duct ligation. They noted a significant increase in expression of the M6P/IGFII receptor as early as 3 days after ligation. There was also a dose-dependent inhibition of fibroblast proliferation by their drug delivery system (Greupink, Bakker et al. 2006). These findings support the theory of competitive inhibition at the M6P/IGFII receptor over that of reduction of dependence on substrate-level phosphorylation as the predominant mechanism by which M6P inhibits fibrosis.

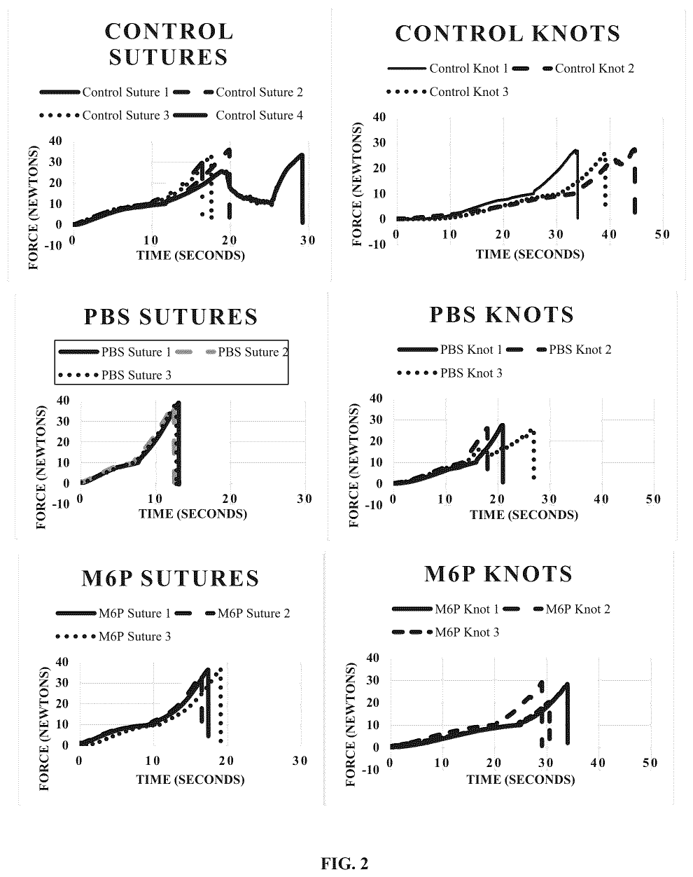

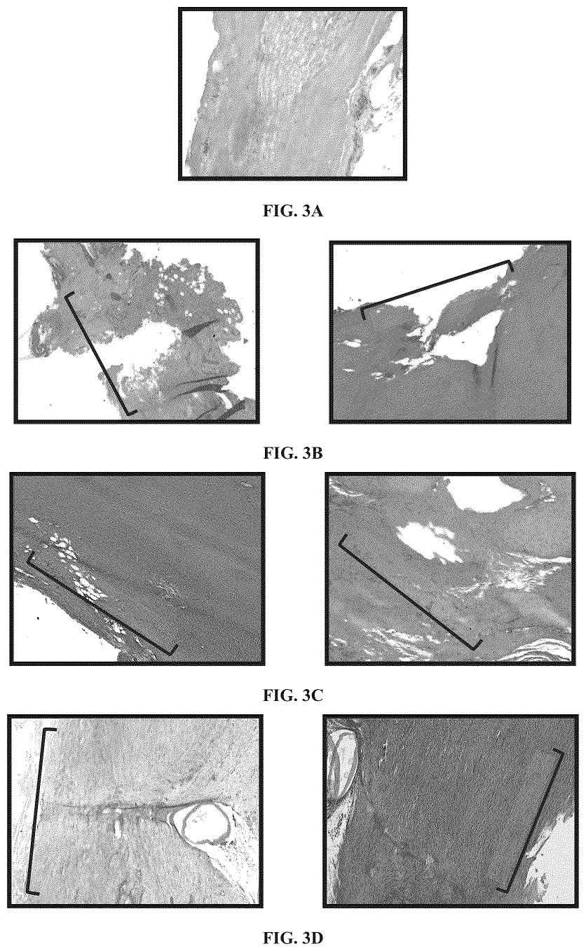

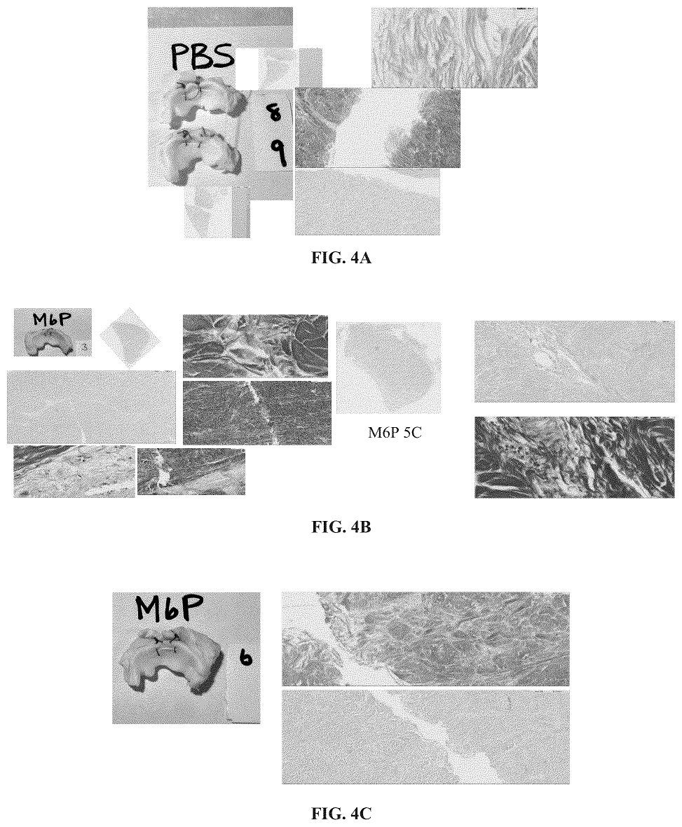

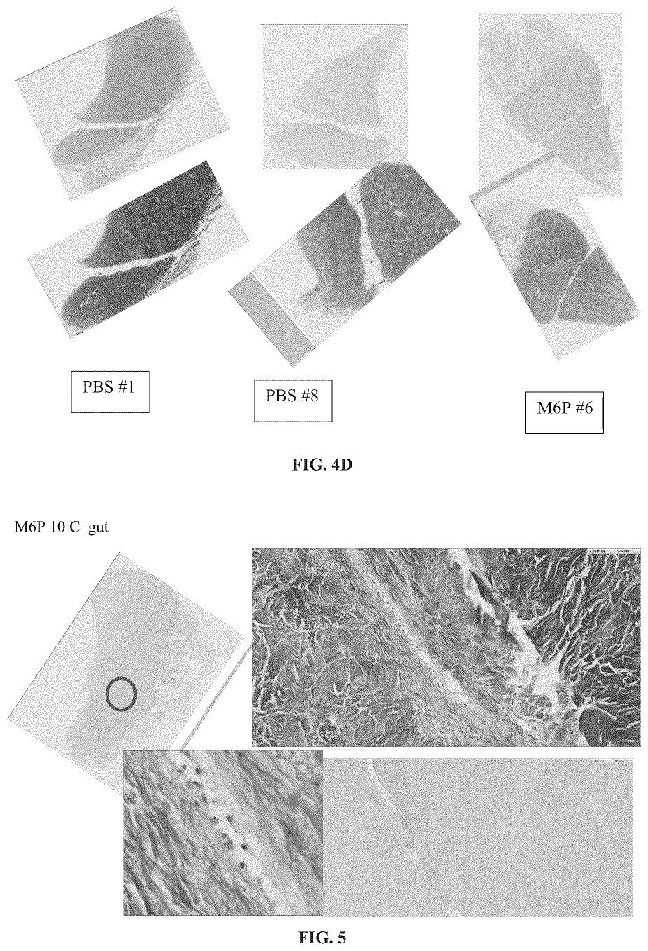

The experiments in this Example cover coating the sutures and evaluating changes in suture mechanical properties. Evaluation of suture physical properties is shown in FIG. 2, while TGA analysis of suture material at 1 year past coating showed control (n=4) mass at 395.28° C. of 87.81%±1.4 and M6P coated (n=4) mass at 395.28° C. of 91.71%±1.04. Additionally, tendon repair (FIGS. 3A-C), meniscal repair (FIGS. 4A-C), and neuroma formation in the presence of M6P coated sutures were evaluated. Tendon repair was evaluated in rat Achilles tendons at 2 and 4 weeks of repair using control (FIG. 3A), M6P direct delivery (FIG. 3B), and M6P sustained delivery (SD) (FIG. 3C). With respect to meniscal repair, referring to FIG. 4A, there was no evidence in any of the PBS treated sutures of increased healing within the time period. Some increased new immature collagen was observed along the cut boarder, but nothing crossing the gap (4 out 4 menisci no growth). Turning to FIG. 4B, in contrast to the PBS treated sutures, the M6P coated sutures showed increased healing within the time period. This increased healing is further evidenced by the difference in tissue formation between the tissue treated with the PBS coated sutures and the single failed M6P coated sutures, which was determined by measurement of the gap width in the tissue (FIG. 4C). More specifically, an average gap width of 835.2 mm±210.88 mm was found for the PBS suture closure, while a gap width of 179.2 mm±55.3 mm was found for the single failed M6P sample. As illustrated in FIG. 5, gut and silk sutures provide a similar type tissue response, which shows that the response would be similar for different types of sutures.

Additional studies were run to show normal tissue formation with minimal tissue adhesion. The coated sutures materials provide a means to enhance normal tissue formation without increasing the risk for fibrosis. An in vivo nerve constriction was performed that normally results in significant adhesion, nerve sprouting, and pain within one week of constriction using the rat as a model. The constriction was made by ligating the sciatic nerve with either 4 saline coated or M6P coated sutures above the truncation and then assessing the animals for pain and gait over a 21 day period post ligation surgery.

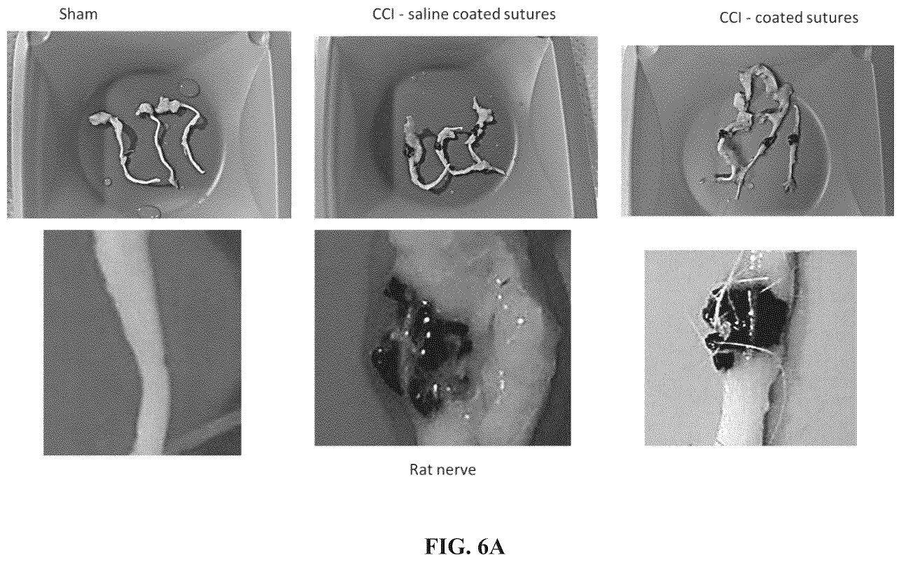

There was significant improvement in the von Frey test for mechanical sensitivity within 1 week of surgery reaching control sham surgical treated animals within two weeks post-surgery. Animals treated with saline coated sutures showed no improvement in mechanical sensitivity for the duration of the study. After 21 days post-surgical ligation, the tissues were harvested and compared for gross visualization of tissue response (FIG. 6A), then histologically (FIG. 6B). The tissue was assessed for six morphological characteristics for the developing neuroma: 1) normal nerve, 2) degenerating axons, 3) axonal sprouts, 4) unorganized bundles of axons, 5) unorganized axon growth into muscles, and 6) unorganized axon growth into fibrotic tissue (neuroma). Visualization of the gross tissue showed that saline coated sutures were completely encapsulated with fibrous tissue, while M6P coated sutures had minimal fibrous tissue associated with the suture after 21 days. In addition, the histology showed striking changes in the saline coated suture group of nerve sprouting, degenerating axons, unorganized bundles of axons, and unorganized axon growth into muscles. Striking differences could be seen in the amount of tissue in close contact with the nerve and the suture in the saline treated group compared with M6P coated suture group. In addition to less adhesion formation, there was also a marked improvement in the gait of the animal by 7 days and within 11 days, toe spreading, foot angle, step angle, and stride width all returned toward baseline and naïve control values which were significantly different from saline coated suture group values.

### Example 2

### In Vitro Experiments

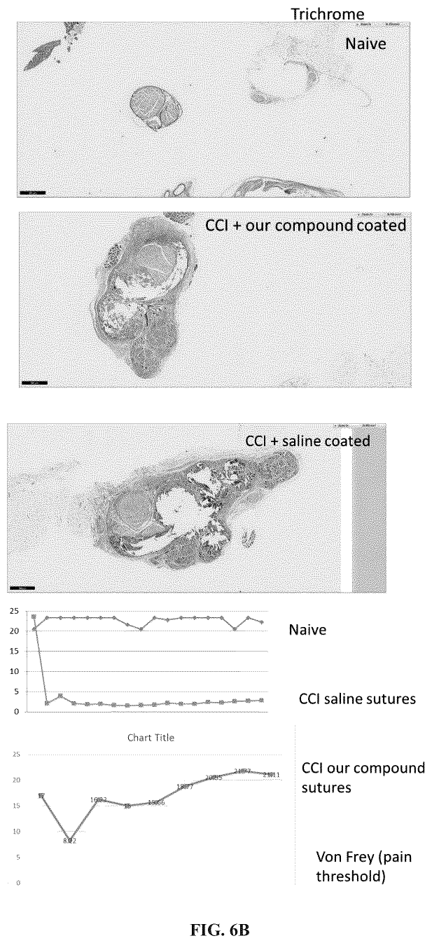

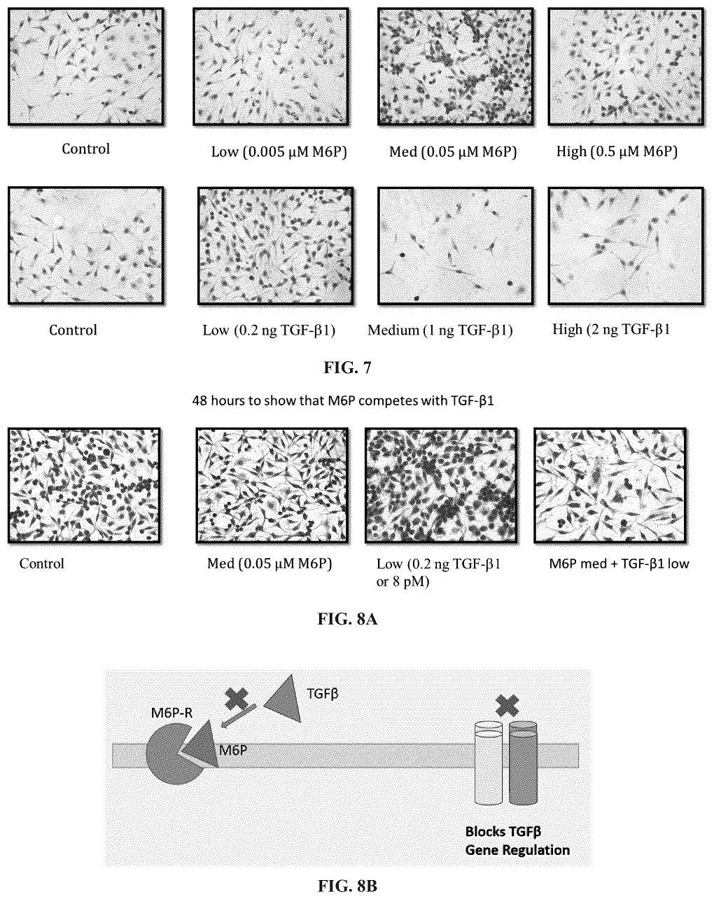

As illustrated in FIG. 7, cellular viability and an increase in the number of lifting off of the coverslip were seen at the higher doses of TGF β (ED₅₀/EC₅₀ 0.01-0.5 ng/ml). Additionally, it was found that 0.05 μM of M6P can enhance cell proliferation of fibroblast cells without evidence of a toxic effect. TGF-β1 enhanced cell proliferation when added at 8 pM, but was toxic to the cells when added at the higher doses. When M6P was added along with TGF-β1 the increased proliferation was eliminated (FIGS. 8A-B).

### Capsule Pore Analysis

Mercury intrusion porosimetry performed by Quantachrome Instruments (Boynton Beach, FL) revealed the following data (Table 8) for a set of 4 matrix-type TCP capsules formed to the same diameter (0.8 mm) and under the same conditions as the capsules implanted in the rats:

| Sample | Mode Pore Diameter (μm) | Pore Volume (cc/g) |
|---|---|---|
| 1 | 1.846 | 0.1126 |
| 2 | 1.870 | 0.1153 |
| 3 | 1.817 | 0.1102 |
| 4 | 1.731 | 0.0943 |

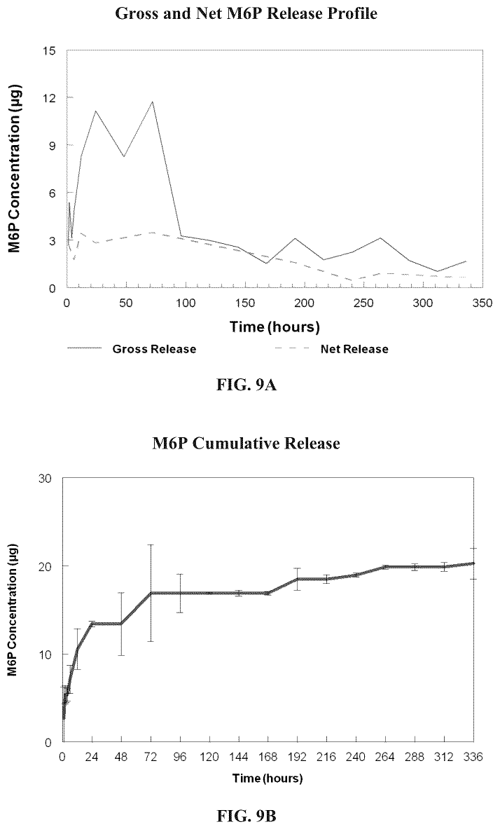

FIGS. 9A-B show release of M6P from TCPL drug delivery device.

All publications, patents, and patent applications mentioned in this specification are herein incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only, and not for the purpose of limitation.

We claim:

1. A tissue repair article, comprising:
a material combined with one or more with a wound healing agents; wherein the wound healing agent comprises
mannose 6 phosphate (M6P), fructose 6 phosphate (F6P), or glucose 6 phosphate (G6P) or a combination thereof; or
a sugar precursor of mannose 6 phosphate (M6P), fructose 6 phosphate (F6P), or glucose 6 phosphate (G6P) or a combination thereof.

2. The article of claim 1, wherein the material is a surgical suture.

3. The article of claim 2, wherein the suture is coated with the wound healing agent.

4. The article of claim 2, wherein the suture is impregnated with the wound healing agent.

5. The article of claim 2, wherein the suture is absorbable when surgically implanted in a subject.

6. The article of claim 2, wherein the suture comprises silk, gut, polypropylene, PDS, poliglecaprone, polyglactin, polyester, steel, monofilament, multifilament, polytetrafluoroethylene, poly (DL-lactide-ε-caprolactone, polyester/polyethylene (PE/PEE).

7. The article of claim 1, wherein the wound healing agent comprises mannose 6 phosphate (M6P), fructose 6 phosphate (F6P).

8. The article of claim 1, wherein the wound healing agent further comprises at least one liposome, amphiphilic polymer, protein, polycaprolactone (PLA), polyglycolide (PGA), mannuronic acid, hydrogel, a hydrogel derived from alginate (seaweed).

9. The article of claim 2, wherein the suture is an incisional wound suture.

10. The article of claim 1, wherein the material is an incisional wound suture and the wound healing agent further comprises mannose.

11. The article of claim 1, wherein the material is a bandage.

12. The article of claim 11, wherein the bandage is cloth, gauze, or an adhesive bandage.

13. The article of claim 1, wherein the material is a bandage, and the wound healing agent further comprises at least one liposome, amphiphilic polymer, protein, polycaprolactone (PLA), polyglycolide (PGA), mannuronic acid, hydrogel, a hydrogel derived from alginate (seaweed), or an analgesic.

14. The article of claim 1, wherein the material is a natural or synthetic woven fabric, or steel mesh.

15. The article of claim 14, wherein the natural or synthetic woven fabric is a multifilamentous polypropylene mesh.

16. The article of claim 1, wherein the wound healing agent exhibits sustained release when in contract with a wound site.

17. The article of claim 2, wherein the suture is coated with a biodegradable layer, and the biodegradable layer is associated with the wound healing agent.

18. The article of claim 1, wherein the wound healing agent is present in an amount of about 25 to 70 mM, preferably about 50 mM.

19. The article of claim 1, wherein the wound healing agent is present in an amount to provide at least three days of sustained release when in contact with an injury site.

20. The article of claim 1, wherein the wound healing agent is present in amount to effectively inhibit TGF-β at a wound site.

21. A method of promoting tissue healing, comprising:
providing a material combined with one or more with a wound healing agents; wherein the wound healing agent comprises mannose 6 phosphate (M6P), fructose 6 phosphate (F6P), or glucose 6 phosphate (G6P) or a combination thereof; or a sugar precursor of mannose 6 phosphate (M6P), fructose 6 phosphate (F6P), or glucose 6 phosphate (G6P) or a combination thereof;
contacting the material to an injury site.

22. The method of claim 21, wherein the material is a suture and the injury site is surgical site on a tendon.
